(19) United States
(12) Patent Application Publication
(10) Pub. No.: US 2026/0306852 A1
(43) Pub. Date: Oct. 8, 2026

(54) SYSTEM AND METHOD FOR PERFORMING A THERAPEUTIC AND/OR DIAGNOSTIC FUNCTION IN A PATIENT

(71) Applicant: BIOTRONIK SE & Co. KG, Berlin (DE)

(72) Inventors: Dirk MUESSIG, West Linn, OR (US); R. Hollis WHITTINGTON, Portland, OR (US); Patrick L. PARISH, Portland, OR (US); Brian M. TAFF, Portland, OR (US)

(73) Assignee: BIOTRONIK SE & Co. KG, Berlin (DE)

(21) Appl. No.: 19/483,433

(22) PCT Filed: May 3, 2024
(86) PCT No.: PCT/EP2024/062193

§ 371 Date: Nov. 11, 2025

Related U.S. Application Data
(60) Provisional application No. 63/466,378, filed on May 15, 2023.

(30) Foreign Application Priority Data
Jun. 7, 2023 (EP) 23177999.2

(51) Int. Cl. A61N 1/365; A61B 5/00; A61N 1/372

(52) U.S. Cl. CPC A61N 1/36578; A61B 5/686; A61B 5/6869; A61N 1/37258

(57) ABSTRACT

A system for performing a therapeutic and/or diagnostic function in a patient includes an implantable medical device configured for implantation in a patient, the implantable medical device including a motion sensor for sensing a motion signal indicative of a motion of the patient. A processing arrangement for processing the motion signal obtained by the motion sensor is configured to process said motion signal by comparing information derived from said motion signal to a defined signature motion pattern in order to identify a myocardial infarction event.

### CROSS-REFERENCE TO RELATED APPLICATIONS

This application is the United States National Phase under 35 U.S.C. § 371 of PCT International Patent Application No. PCT/EP2024/062193, filed on May 3, 2024, which claims the benefit of European Patent Application No. 23177999.2, filed on Jun. 7, 2023, and U.S. Provisional Patent Application No. 63/466,378, filed on May 15, 2023, the disclosures of which are hereby incorporated by reference herein in their entireties.

### TECHNICAL FIELD

The instant invention generally relates to a system for performing a therapeutic and/or diagnostic function in a patient and to a method for operating such a system.

### BACKGROUND

A system of the type concerned herein comprises an implantable medical device configured for implantation in a patient, the implantable medical device comprising a motion sensor for sensing a motion signal indicative of a motion of the patient. A processing arrangement is configured for processing signals obtained by the motion sensor.

An implantable medical device of this kind may for example be a pacemaker, an implantable cardioverter defibrillator, a sensor device such as a bio-sensor, or a monitoring device. The implantable medical device herein is for example configured to sense electrocardiogram signals.

For example, the implantable medical device may be a monitoring device which is configured to record electrocardiogram signals and to communicate recorded electrocardiogram signals or information derived from recorded electrocardiogram signals to an external device in the context of a home monitoring system.

An implantable medical device as for example described in EP 3 278 836 B1 may for example comprise a housing and an arrangement of electrode poles arranged on the housing. The electrode poles herein are arranged on the housing of the implantable medical device such that the electrode poles are aligned along a longitudinal axis along which the implantable medical device extends. The electrode poles may for example be formed by housing segments which are made from an electrically conductive material such as a metal material and are exposed to the outside such that they may be brought into electrical contact with surrounding tissue in order to establish an electrical coupling to the tissue in an implanted state of the implantable medical device.

An implantable medical device as e.g. used in a home monitoring system shall allow for a reliable monitoring of a physiological state of a patient. In particular, using the implantable medical device it shall be possible to reliably detect an abnormal cardiac state. If an abnormality is detected, the implantable medical device shall be enabled to communicate with for example an external device of a home monitoring system, e.g. in order to trigger a message to a service center to alert medical personnel of a potential need for attention.

Among those cardiac abnormalities which make a close, reliable monitoring desirable is a state of an acute myocardial infarction. Acute myocardial infarctions represent a leading cause of death in the developed world resulting in, nowadays, an estimated 3,000,000 lives lost worldwide per year with more than 1,000,000 lives lost in the US alone. As in a state of acute myocardial infarction immediate medical attention to a patient is crucial to improve the chances of treating the myocardial infarction, it is desired to improve approaches to support predicting, recognizing and rapidly reporting the occurrence of sudden ischemic cardiac conditions.

The present disclosure is directed toward overcoming one or more of the above-mentioned problems, though not necessarily limited to embodiments that do.

### SUMMARY

It is an object of the instant invention to provide a system for performing a therapeutic and/or diagnostic function in a patient and a method for operating such a system which allow for a monitoring of a cardiac condition to identify a myocardial infarction event in a timely manner.

In one aspect, a system for performing a therapeutic and/or diagnostic function in a patient comprises an implantable medical device configured for implantation in a patient, the implantable medical device comprising a motion sensor for sensing a motion signal indicative of a motion of the patient, particularly the patient's body or a part thereof. The implantable medical device furthermore comprises a processing arrangement for processing the motion signal obtained by the motion sensor. The processing arrangement is configured to process the motion signal by comparing information derived from the motion signal to a defined signature motion pattern in order to identify a myocardial infarction event.

The system comprises an implantable medical device configured for implantation in a patient. The implantable medical device comprises a motion sensor which is configured, during operation of the implantable medical device in the patient, to sense a motion signal indicative of a motion of the patient. Particularly, the motion signal as sensed by the motion sensor may be indicative of a motion of a body part, for example of the patient's chest, or of a motion of the patient as a whole.

The implantable medical device may for example be a pacemaker, an implantable cardioverter defibrillator, a sensor device such as a bio-sensor, or a monitoring device. The implantable medical device herein is for example configured to sense electrocardiogram signals.

Based on the motion signal as sensed by the motion sensor of the implantable medical device a processing arrangement of the system is configured to identify a myocardial infarction event. In particular, the processing arrangement is configured to process the motion signal by comparing information derived from the motion signal to a defined signature motion pattern. In order to identify the occurrence of a myocardial infarction event, the processing arrangement derives information from the motion signal, which may be compared to reference information as defined for a defined signature motion pattern. If based on the comparison of the information derived from the motion signal to the defined signature motion pattern a match is identified, it is concluded that a myocardial infarction event has occurred.

Based on the motion signal, hence, a pattern matching is carried out by the processing arrangement. If a pattern identified according to the motion signal matches a defined signature motion pattern indicative of a myocardial infarction event, it is concluded that a myocardial infarction event is present.

In one embodiment, the implantable medical device comprises a processing circuitry which implements at least a portion of the processing arrangement. In general, the processing may be conducted entirely within the implantable medical device in order to identify a myocardial infarction event by the implantable medical device. In this case the processing arrangement is implemented by the processing circuitry of the implantable medical device. In other embodiments, the processing arrangement is in part or entirely implemented externally to the implantable medical device, in which case the implantable medical device for example only pre-processes a motion signal obtained by the motion sensor and forwards the motion signal to the processing arrangement external to the implantable medical device. The processing for identifying a myocardial infarction event hence takes place substantially outside of the implantable medical device, for example in an external device communicating with the implantable medical device for example in the context of a home monitoring system, or on a server device stationed at a remote location, such as a home monitoring service center connected to the external device and via the external device to the implantable medical device via a public communication network, such as the Internet.

In one embodiment, the system comprises an external device configured for operation external to the patient. The implantable medical device is configured to communicate data relating to the motion signal or relating to information derived from the motion signal to the external device. For example, the implantable medical device may communicate the motion signal as such as a raw data signal to the external device for processing in the external device or in another device connected to the external device. In other embodiments, the implantable medical device may substantially carry out the processing for identifying a myocardial infarction event and may forward information relating to an identified myocardial infarction event to the external device, in which case at least a majority of the processing is carried out by the implantable medical device and only a post-processing is carried out by the external device or by a device connected to the external device.

In one embodiment, the system comprises a server device configured to communicate with the external device for transferring information to and/or receiving information from the external device. The server device, for example a home monitoring service center in the context of a home monitoring system, is configured to process information derived from the motion signal to identify a myocardial infarction event and/or to process information relating to an identified myocardial infarction event. A substantial amount of the processing may be carried out by the server device, in which case for example raw data of the motion signal or pre-processed information indicative of information derived from the motion signal is forwarded to the server device, which carries out further processing for identifying a myocardial infarction event. In another embodiment, the server device may receive information relating to an identified myocardial infarction event, as it has been determined by the implantable medical device or by the external device, in order to post-process such information, for example to trigger an action in response to the identification of a myocardial infarction event.

In one embodiment, the motion sensor of the implantable medical device is an accelerometer. The accelerometer may be a single-axis accelerometer which can detect the magnitude and direction of an acceleration along a single axis. In another embodiment, the accelerometer may be a multi-axis accelerometer that can detect the magnitude and direction of an acceleration along multiple axes.

From an acceleration signal as detected by the accelerometer a velocity signal or a position signal may be derived. An acceleration signal, a velocity signal or a position signal, in the context of the instant text, are each understood to represent a motion signal indicative of a motion of the patient.

In one embodiment, the processing arrangement is configured to store a multiplicity of defined signature motion patterns for comparison to the information derived from the motion signal. In particular, the processing arrangement may store multiple templates, each template relating to a defined signature motion pattern indicative of a particular, characteristic motion pattern correlating with a myocardial infarction event. In particular, when a myocardial infarction occurs, this may come with a typical, identifiable motion of a patient. For example, in the case of an acute myocardial infarction a patient may clutch her chest, may bend over and may collapse to the floor, which may be followed by a lengthy pause in which the body of the patient is substantially motionless. Such a motion pattern may be identifiable according to a defined signature motion pattern, such that when a motion signal is sensed a pattern as identified within the motion signal may be matched to a defined signature motion pattern indicative of a myocardial infarction event. Accordingly, based on a pattern matching the occurrence of a myocardial infarction event may be identified.

Multiple different signature motion patterns may be defined such that based on a pattern matching a sensed motion pattern may be matched to a multiplicity of different defined signature motion patterns. If a match with one signature motion pattern is found a myocardial infarction event is identified.

In one embodiment, the processing arrangement is configured to determine, in order to derive information from the motion signal, a set of characteristic metrics values from the motion signal and to compare the set of characteristic metrics values to a set of reference values defined for a defined signature motion pattern. For example, based on a motion signal, an amplitude of a motion event, an integral under a curve relating to a motion event, a time duration of a motion event, a time duration between a motion event and a subsequent motion event, a relation of amplitudes of a motion event and a subsequent motion event, the occurrence and time duration of a zero-motion phase in which no movement of the patient occurs and/or a rate of a time-varying periodic motion signal may be determined. Characteristic metrics values as determined for the motion signal may be compared to a set of corresponding reference values corresponding to a defined signature motion pattern. If a match is found between the characteristic metrics values and the set of reference values, a myocardial infarction event is identified.

A match between a characteristic metrics value and an associated reference value herein may be found if the particular characteristic metrics value does not deviate from the reference value by more than a certain margin.

A match between a set of characteristic metrics values and a set of reference values defined for a defined signature motion pattern may be identified if at least for a subset of the set of characteristic metrics values a match with the corresponding reference values is identified.

Generally, one or multiple signature motion patterns may be identified empirically by an expert analysis of data in order to derive characteristic information which may be indicative of a myocardial infarction event in a motion signal. In another embodiment, one or multiple signature motion patterns may be automatically identified based on a data collection which may be used as training data to train a model for identifying characteristic signature motion patterns.

In one embodiment, the system is configured to define the defined signature motion pattern based on training data using an artificial intelligence model. Generally, an artificial intelligence model may be fed with a large amount of training data, in particular motion data of patients for which myocardial infarction events have been identified, e.g. by physicians in clinical environments for patients suffering from a myocardial infarction. For example, training data may be collected by using implantable medical devices, in particular motion data obtained by the implantable medical devices, and by associating motion data with known myocardial infarction events.

An amount of training data useful for identifying signature motion patterns may evolve over time subject to analytics applied to ongoing data collection efforts applied across a pool of patients implanted with such implantable medical devices who experienced known and confirmed myocardial infarction events. The collection of information may be supported by any one of multiple possible avenues including but not limited to mechanisms that relay the status of patient and device conditions to wearables, mobile devices, or bedside units that can pass data further on to centralized repositories. Artificial intelligence-based surveys of a growing body of information collected from the pool of patients may serve to enhance the predictability and accuracy of signature motion patterns usable for a pattern matching for identifying a myocardial infarction event.

Signature motion patterns as determined by an artificial intelligence model, for example on a server device, may be uploaded to an implantable medical device in a periodic fashion, such that a set of signature motion patterns is periodically updated on the implantable medical device.

In one embodiment, the implantable medical device comprises an electrode arrangement formed by an arrangement of electrode poles for sensing electrocardiogram signals. Electrode poles may for example be formed on a housing of the implantable medical device. Alternatively or in addition, one or multiple electrode poles may be formed on an electrode lead extending from a housing of the implantable medical device, the electrode lead extending for example into the patient's heart, for example into the right or left ventricle of the patient's heart. By means of the electrode arrangement electrical signals relating to cardiac activity may be sensed in order to record electrocardiogram signals, which may be processed in order to derive information indicative of a cardiac state.

In one embodiment, the processing arrangement is configured to identify the myocardial infarction event based on the comparison of information derived from the motion signal to a defined signature motion pattern and in addition based on at least one of respirational information and electrocardiogram information. In addition to matching information derived from the motion signal to a defined signature motion pattern, information stemming from further input streams relating to for example a respirational state of the patient or relating to electrocardiogram signals may be taken into account in order to identify a myocardial infarction event.

Respirational information indicative of the respiration of the patient may for example be identified based on the motion signal obtained by the motion sensor. Particularly, the motion signal may be indicative of and/or include the posture of the patient. In another embodiment, respirational information may be derived for example from bio-impedance information sensed by the implantable medical device or by another device which is in communication connection with the implantable medical device.

For example, a myocardial infarction event may coincide with a particular respirational signature, indicative for example of heavy breathing, which may be identified based on respirational information and may be taken into account for identifying a myocardial infarction event. In addition, a myocardial infarction event may correlate with a sudden increase in heart rate not affiliated with patient exercise conditions, the heart rate for example being determined based on electrocardiogram information. Hence, also heart rate information may be taken into account for identifying a myocardial infraction event.

Generally, by taking further information into account, in addition to the pattern matching of a sensed motion signal to a defined signature motion pattern, a model for identifying a myocardial infarction event may be rendered more robust.

In one embodiment, the processing arrangement is configured to trigger an alert action based on the identification of a myocardial infarction event. Generally, if a myocardial infarction event is identified, the processing arrangement may trigger an alert message for example to healthcare personnel in order to alert the healthcare personnel of a worsening of the condition of the patient.

For example, for triggering an alert action, the processing arrangement may be configured to generate, as the alert action, an alarm message to an emergency facility, the message including information for identifying the patient to the emergency facility. For example, an automatic call to an emergency hotline, e.g. an emergency phone number such as “911” in the US, may be initiated, such that an emergency facility is alarmed to provide immediate attention to the patient suffering from an acute myocardial infraction. A message to the emergency facility may for example include the time of identification of the myocardial infarction event, the name of the patient and a physical location of the patient, such that emergency treatment may be provided to the patient.

In addition, a message for example to identified individuals, such as family members or friends may be triggered in order to alert such individuals.

In another aspect, a method for operating a system for performing a therapeutic and/or diagnostic function in a patient comprises: providing an implantable medical device configured for implantation in a patient, the implantable medical device comprising a motion sensor for sensing a motion signal indicative of a motion of the patient, particularly a motion of a body part, for example of the patient's chest, or of a motion of the patient as a whole; providing a processing arrangement for processing a motion signal obtained by the motion sensor; and processing, using the processing arrangement, a motion signal sensed by the motion sensor by comparing information derived from the motion signal to a defined signature motion pattern in order to identify a myocardial infarction event.

The advantages and advantageous embodiments as described above for the system equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic drawing of an implantable medical device implanted in a patient;

FIG. 2 shows a schematic drawing of an embodiment of an implantable medical device comprising an arrangement of electrode poles;

FIG. 3 shows a schematic drawing of another embodiment of an implantable medical device;

FIG. 4 shows an example of a motion signal as obtained with a motion sensor of an implantable medical device;

FIG. 5A shows an example of another motion signal obtained by the motion sensor;

FIG. 5B shows a heart rate signal obtained based on electrocardiogram signals sensed by the implantable medical device;

FIG. 6 shows a schematic drawing of an artificial intelligence model in order to derive signature motion patterns for conducting a pattern matching for identifying a myocardial infarction event; and

FIG. 7 shows a schematic drawing of a workflow for triggering an alert action based on an identified myocardial infarction event.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

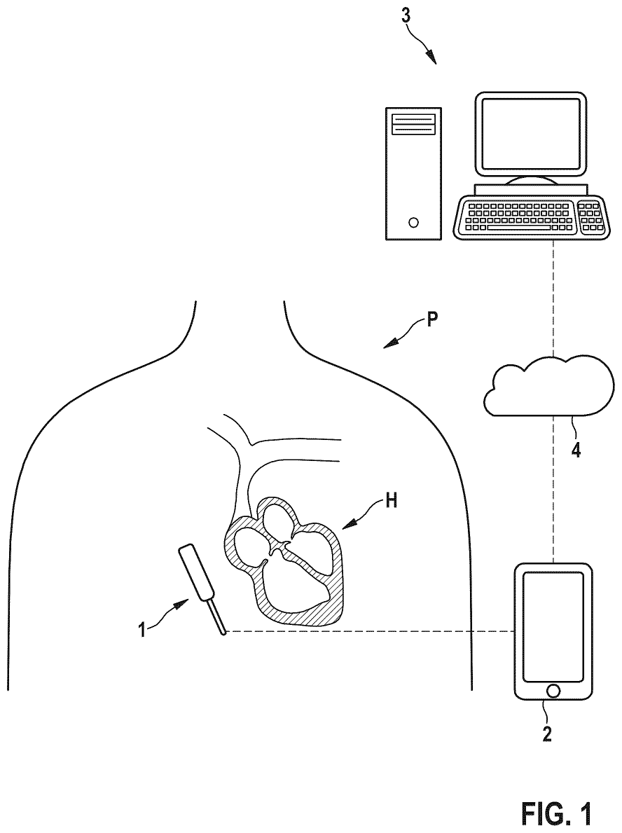

Referring to FIG. 1, in one embodiment a system comprises an implantable medical device 1 implanted (for example subcutaneously) into a patient for serving a therapeutic and/or diagnostic function. The implantable medical device 1 may for example be implanted subcutaneously into a patient P for monitoring cardiac activity of the patient's heart H. The implantable medical device 1, for this, comprises an arrangement of electrode poles which are used to couple to surrounding tissue and to sense electrocardiogram signals originating from the heart H.

The system furthermore comprises an external device 2 external to the patient P and being in communication connection with the implantable medical device 1. The external device 2 may be in connection, via a public communication network 4, with a server device 3, for example a home monitoring service center, in the context of a home monitoring system.

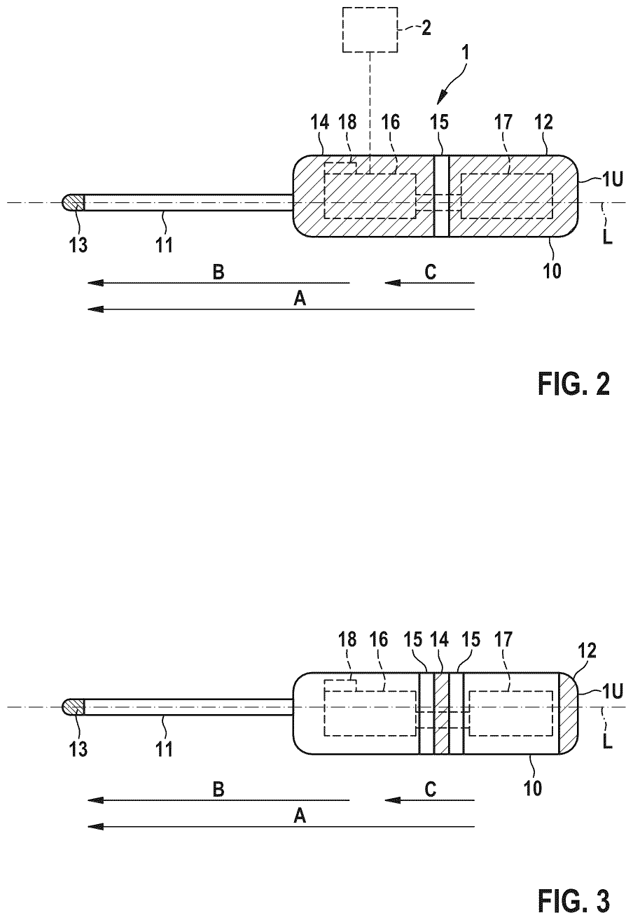

Referring now to FIG. 2, in one embodiment the implantable medical device 1 comprises a housing 10 formed e.g. by different housing segments, the housing 10 enclosing and encapsulating a processing module 16 formed by electronic circuitry and a battery module 17. In particular, a first housing segment may receive and enclose the processing module 16, whereas a second housing segment receives and encloses the battery module 17. Another housing segment 11 longitudinally extends from the first and second housing segments and forms a header portion having reduced cross-sectional dimensions with respect to the other housing segments.

In the embodiment of FIG. 2, a first electrode pole 12 is formed by the housing segment enclosing the battery module 17, a second electrode pole 13 is arranged at a far end of the housing segment 11 forming the header portion, and a third electrode pole 14 is formed by the housing segment enclosing the processing module 16. The implantable medical device 1 with its housing 10 generally extends along a longitudinal axis L, the electrode poles 12, 13, 14 being aligned along the longitudinal axis L and being axially displaced with respect to one another along the longitudinal axis L. The electrode poles 12, 13, 14 herein are electrically separated from one another, an electrically insulating segment 15 being arranged in between the electrode poles 12, 14 formed on the main housing portion and the header portion formed by the housing segment 11 separating the electrode pole 13 from the other two electrode poles 12, 14.

In the embodiment of FIG. 2, the electrode poles 12, 13, 14 may be formed by portions of the housing 10 itself, the housing 10 being made for example from an electrically conductive material, in particular a metal material. By exposing portions of the housing 10 towards the outside, the electrode poles 12, 13, 14 are formed and may electrically contact with surrounding tissue in order to establish a coupling between the electrode poles 12, 13, 14 to the surrounding tissue.

Referring now to FIG. 3, in another embodiment the first electrode pole 12 is formed at an end of a housing segment of the housing 10 encapsulating the battery module 17, whereas the electrode pole 14 is formed by an electrode element which is electrically insulated from other portions of the housing 10 by insulating segments 15. For example, a multilayered pole element may be employed for forming the electrode pole 14, as it is described for example in EP 3 278 836 B1. The electrode pole 13 again is formed at a far end of the housing segment forming the header portion 11.

In any of the embodiments of FIGS. 2 and 3, using the arrangement of electrode poles 12, 13, 14, electrocardiogram signals may be received and processed by the processing module 16. Based on the processing, a communication with an external device 2 may be established, for example to transmit alert messages to the external device 2 and on to the server device 3 for example within the context of a home monitoring system for monitoring a physiological state of the patient P.

The different electrode poles 12, 13, 14 herein define signal reception vectors A, B, C by means of which electrocardiogram signals may be received using pairs of associated electrode poles 12, 13, 14. In particular, a first signal reception vector A is formed between the first electrode pole 12 and the second electrode pole 13, a second signal reception vector B is formed between the third electrode pole 14 and the first second electrode pole 13, and a third signal reception vector C is formed between the first electrode pole 12 and the third electrode pole 14. As the first electrode pole 12 and the second electrode pole 13 are arranged at opposite ends of the housing 10, the associated signal reception vector A is longer than the other two signal reception vectors B, C.

The different electrode poles 12, 13, 14 form different pairs of electrode poles 12, 13, 14 spanning different signal reception vectors A, B, C. By means of the different signal reception vectors A, B, C different electrocardiogram signals may be received and may be processed in a multi-channel processing.

It shall be noted herein that the implantable medical device 1 may be a monitoring device (as schematically shown in FIGS. 2 and 3), a pacemaker device, a defibrillator device or any other implantable medical device configured for implantation into a patient P. The instant text in particular is not limited to a monitoring device configured for implantation outside of a patient's heart H.

The implantable medical device 1 comprises a motion sensor 18, which may be implemented by an accelerometer, in particular a single-axis accelerometer or multi-axis accelerometer configured for sensing an acceleration signal indicative of a motion of the patient P. Using the accelerometer, an acceleration signal may be obtained, from which a velocity signal and/or a position signal may be derived. Within this text, an acceleration signal, a velocity signal and a position signal generally are referred to as motion signal indicative of a motion of the patient P.

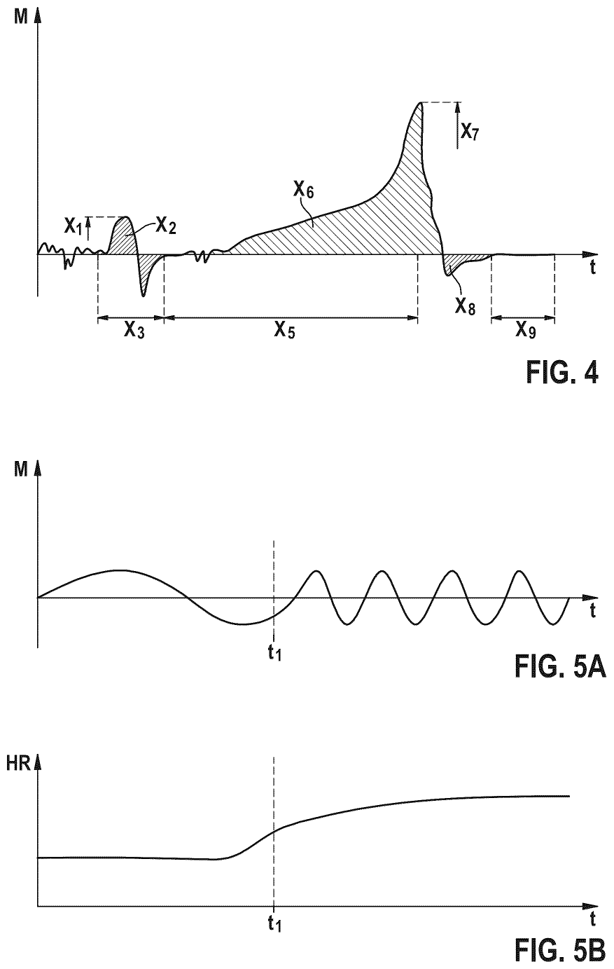

Referring now to FIG. 4, the system including the implantable medical device 1 and the external device 2 shall be configured for identifying a myocardial infarction event based on a motion signal M as obtained using the motion sensor 18 of the implantable medical device 1. A motion signal M herein may be processed in order to derive information from the motion signal M, wherein the processing may be carried out by the processing circuitry 16 of the implantable medical device 1, or by the external device 2 or the server device 3 which for this purpose may receive information relating to the motion signal M from the implantable medical device 1.

In particular, from a motion pattern in a motion signal M characteristic metrics values X1 to X9 may be determined in order to characterize the motion pattern within the motion signal M.

Herein, a first characteristic metric X1 may relate to a maximum positive amplitude of a first signal waveform. Another, second characteristic metric X2 may relate to an area under a curve relating to the first signal waveform. Yet another characteristic metric X3 may relate to a time duration of the first signal waveform. Yet another characteristic metric X4 may relate to a maximum negative amplitude of the first signal waveform. Yet another characteristic metric X5 may relate to a time duration between a first signal waveform and a peak of a subsequent signal waveform. Yet another characteristic metric X6 may relate to an area under a positive portion of a curve relating to the second signal waveform. Yet another characteristic metric X7 may relate to a maximum amplitude of a peak of the second signal waveform. Yet another characteristic metric X8 may relate to an area under a negative portion of the curve relating to the second signal waveform. Yet another characteristic metric X9 may relate to a time span subsequent to the second signal waveform in which no motion is detected (zero-amplitude signal).

Based on the motion signal M a pattern matching may take place in which, based on a processing of the motion signal M, characteristic metrics values X1 to X9 as identified for the motion signal M are compared to corresponding reference values is identified for a defined signature motion pattern. Based on a pattern in a sensed motion signal M and based on a matching of the pattern to a defined signature motion pattern a myocardial infarction event may be identified, based on the assumption that the defined signature motion pattern is indicative of a characteristic motion pattern in case of an occurrence of a myocardial infarction event.

For example, in the acute case of a myocardial infarction event a patient may exhibit a particular kind of motion. For example, the patient may clutch her chest, may bend over, may collapse to the floor, may hit the floor and may be motionless after collapsing. This will result in a particular motion pattern, which may be identified based on a motion signal M as sensed using the motion sensor 18 of the implantable medical device 1.

In particular, the motion sensor 18 of the implantable medical device 1 may sense motion of body parts, such as the patient's heart and the patient's chest in the vicinity of the implantable medical device 1. In addition, the motion sensor 18 may sense motion indicative of a movement of the patient as a whole, for example a falling movement. Based on a processing of a motion signal M as obtained using the motion sensor 18, hence, particular motion patterns may be identified and may be matched to defined signature motion patterns associated with a myocardial infarction event. If a match is found, it is identified that a myocardial infarction event is present, such that a particular, defined action may be triggered.

Referring now to FIGS. 5A and 5B, in addition to processing a motion signal M to match a pattern of the motion signal M to a defined signature motion pattern indicative of a movement of the patient as a whole, for example a clutching movement, a collapsing movement or a motionless state, based on a motion signal also respiration information may be derived. For example, in case of a myocardial infarction event a motion signal M may indicate an increase in a respirational rate, as visible in FIG. 5A, showing a normal breathing with a rather slow variation of movement prior to a time t1 and subsequent to time t1 a substantially faster variation in the motion signal M indicative a substantially faster respirational rate. In addition, a heart rate HR may be determined based on electrocardiogram signals as sensed using the electrode arrangement of the electrode poles 12, 13, 14 of the implantable medical device 1. If an increase in heart rate HR is observed at the same time as an increase in the respirational rate is sensed, this may confirm the presence of a myocardial infarction event in addition to the matching of a motion signal M with a defined signature motion pattern.

A set of multiple defined signature motion patterns may be stored within the system, for example within the processing circuitry 16 of the implantable medical device. During operation, a motion signal M may be analyzed with respect to a pattern matching of a particular sensed motion pattern to a defined signature motion pattern, and if a match is detected, a myocardial infarction event is identified.

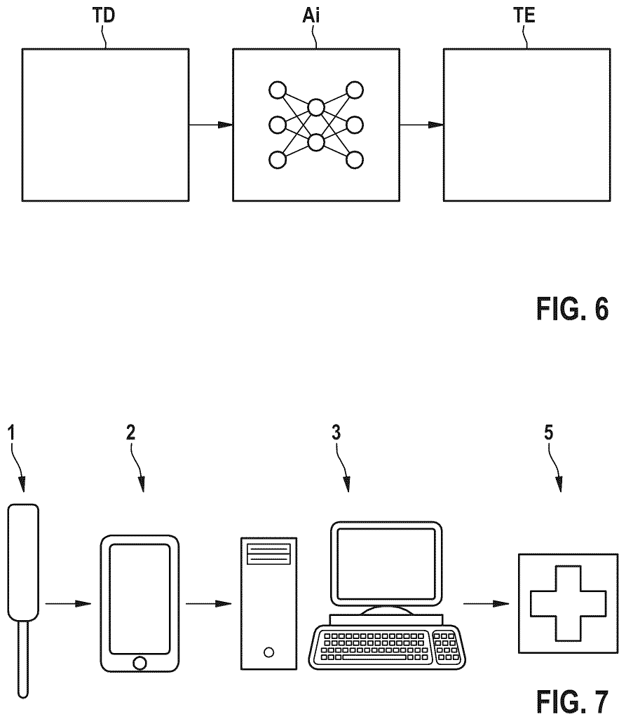

Herein, the set of multiple defined signature motion patterns may be determined using an artificial intelligence model AI, as schematically illustrated in FIG. 6. For determining the signature motion patterns the artificial intelligence model AI may be fed with training data TD, the training data TD for example including motion signals obtained from a large number of implantable medical devices implanted in a pool of patients for which myocardial infarction events have occurred and have been identified for example by the automatic processing of signals or by a confirmation of a physician correspondingly entered into the system. The training data TD may be collected over a large pool of patients, wherein the amount of training data TD may continuously grow with a growing population of implantable medical devices within the patients and a correlation of motion signals with known and confirmed myocardial infarction events. For example, the implantable medical devices may relay information relating to motion signals and myocardial infarction events (using a triggered, periodical or continuous reporting) to wearables, mobile devices, or bedside units that can pass data on to centralized repositories. Based on a data collection on those centralized repositories the artificial intelligence model AI may continuously be refined, and signature motion patterns may be determined as templates TE.

Hence, based on a processing of training data TD using the artificial intelligence model AI signature motion patterns TE indicative of characteristic motion patterns relating to myocardial infarction events may be identified. Such signature motion patterns may be made usable within a system as shown in FIG. 1, in particular on the implantable medical device 1, the external device 2 and/or the server device 3, for processing motion data M sensed in operation of the implantable medical device 1, such that a myocardial infarction event may be identified based on a pattern matching of sensed motion signals M with defined signature motion patterns as defined by the artificial intelligence model AI.

In case a myocardial infarction event is identified using the system of FIG. 1, an alert action may be triggered. For example, an alert message may be sent to healthcare personnel in order to alert the healthcare personal of the myocardial infarction event at the patient.

Alternatively or in addition, an alert message may be sent for example to an emergency facility 5, as illustrated in FIG. 7, such that an emergency treatment may be provided to the patient. For example, a message to an emergency hotline, such as an emergency phone line (“911” in the US), may be triggered, such that emergency treatment may be provided to the patient. The sending of the alert message may be initiated by the server device 3, which receives information relating to an identified myocardial infarction event from an external device 2 that is in communication connection with the implantable medical device 1, or which receives motion signal data via the external device 2 from the implantable medical device 1 and processes the motion data to identify a myocardial infarction event.

In addition, identified individuals, such as family members or friends of the patient, may be notified by alert messages.

An alert message may be triggered automatically in case of identification of a myocardial infarction event. Emergency attention hence may be provided to the patient without relying upon the presence and goodwill of bystanders to the patient, and without relying on a sufficient capacity of the patient to by herself trigger an alert in case of a myocardial infarction event.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### List of Reference Numerals

1 Implantable medical device
10 Housing
11 Header segment
12 Electrode pole
13 Electrode pole
14 Electrode pole
15 Electrically insulating segment
16 Processing module
17 Battery module
18 Motion sensor
2 External device
3 Server device
4 Public communication network
5 Emergency facility
A, B, C Signal reception vector
AI Artificial Intelligence (AI) model
TD Training data
TE Matching template
X1 . . . X9 Characteristic metric

1. A system for performing a therapeutic and/or diagnostic function in a patient, comprising:
an implantable medical device configured for implantation in a patient, the implantable medical device comprising a motion sensor for sensing a motion signal indicative of a motion of a body part of the patient, particularly the patient's chest, or of a motion of the patient as a whole; and
a processing arrangement for processing the motion signal obtained by the motion sensor;
wherein the processing arrangement is configured to process said motion signal by comparing information derived from said motion signal to a defined signature motion pattern in order to identify a myocardial infarction event.

2. The system according to claim 1, wherein the implantable medical device comprises a processing circuitry which implements at least a portion of the processing arrangement.

3. The system according to claim 1, comprising an external device configured for operation external to the patient, wherein the implantable medical device is configured to communicate data relating to said motion signal or relating to information derived from said motion signal to the external device.

4. The system according to claim 3, comprising a server device configured to communicate with said external device for transferring information to and/or receiving information from said external device, wherein the server device is configured to process information derived from said motion signal to identify a myocardial infarction event and/or to process information relating to an identified myocardial infarction event.

5. The system according to claim 1, wherein the motion sensor is an accelerometer.

6. The system according to claim 1, wherein the processing arrangement is configured to store a multiplicity of defined signature motion patterns for comparison to said information derived from said motion signal.

7. The system according to claim 1, wherein the processing arrangement is configured to determine, to derive said information derived from said motion signal, a set of characteristic metrics values from said motion signal and to compare the set of characteristic metrics values to a set of reference values defined for said defined signature motion pattern.

8. The system according to claim 1, wherein the system is configured to define said defined signature motion pattern based on training data using an artificial intelligence model.

9. The system according to claim 8, wherein the training data includes information obtained from a multiplicity of implantable medical devices.

10. The system according to claim 1, wherein the implantable medical device comprises an electrode arrangement formed by an arrangement of electrode poles for sensing electrocardiogram signals.

11. The system according to claim 1, wherein the processing arrangement is configured to identify said myocardial infarction event based on said comparison and in addition based on at least one of respirational information and electrocardiogram information.

12. The system according to claim 11, wherein the implantable medical device is configured to obtain said respirational information based on said motion signal sensed by the motion sensor.

13. The system according to claim 1, wherein the processing arrangement is configured to trigger an alert action based on the identification of a myocardial infarction event.

14. The system according to claim 13, wherein the processing arrangement is configured to generate, as said alert action, an alarm message to an emergency facility, said message including information for identifying the patient to the emergency facility.

15. A method for operating a system for performing a therapeutic and/or diagnostic function in a patient, the method comprising:
providing an implantable medical device configured for implantation in a patient, the implantable medical device comprising a motion sensor for sensing a motion signal indicative of a motion of a body part of the patient, particularly the patient's chest, or of a motion of the patient as a whole;
providing a processing arrangement for processing a motion signal obtained by the motion sensor; and
processing, using the processing arrangement, a motion signal sensed by the motion sensor by comparing information derived from said motion signal to a defined signature motion pattern in order to identify a myocardial infarction event.
